(19) United States
(12) Patent Application Publication
(10) Pub. No.: US 2016/0175544 A1
(43) Pub. Date: Jun. 23, 2016

(54) SINGLE USE INJECTION SYSTEM

(71) Applicant: Marc Andrew Koska, East Sussex (GB)

(72) Inventor: Marc Andrew Koska, East Sussex (GB)

(21) Appl. No.: 14/575,635

(22) Filed: Dec. 18, 2014

(51) Int. Cl. A61M 5/46; A61B 17/20; A61M 37/00

(52) U.S. Cl. CPC A61M 5/46; A61M 37/00; A61B 17/205

(57) ABSTRACT

The invention is a single use intradermal injection device capable of intradermal delivery an agent (e.g., vaccine, drug, medicament, etc.) in a controlled manner and without requiring specialized skill in administering delivery of such agent. The injection device is configured to be filled on-site and in the field with a microdose of an agent, while remaining sterile and preventing the potential for contamination during the filling process. The injection device is further configured to be rendered incapable of reuse following its delivery of the agent to a patient, thereby preventing reuse of the device and reducing the risk of the spreading blood-borne diseases through reuse.

### FIELD OF THE INVENTION

The present invention generally relates to delivery devices for delivering substances, such as medicaments, and, more particularly, to a single use intradermal injection device that is rendered incapable of reuse following its intended use of delivering a therapeutic agent to a patient.

### BACKGROUND

Every year, millions of people become infected and die from a variety of diseases, some of which are vaccine-preventable. Although vaccination has led to a dramatic decline in the number of cases of several infectious diseases, some of these diseases remain quite common. In many instances, large populations of the world, particularly in developing countries, suffer from the spread of vaccine-preventable diseases due to ineffective immunization programs, either because of poor implementation, lack of affordable vaccines, or inadequate devices for administering vaccines, or combinations thereof.

Some implementations of immunization programs generally include administration of vaccines via a typical reusable syringe. However, in many situations, particularly in developing countries, the administration of vaccines occur outside of a hospital and may be provided by a non-professional, such that injections are given to patients without carefully controlling access to syringes. The use of reusable syringes under those circumstances increases the risk of infection and spread of blood-borne diseases, particularly when syringes, which have been previously used and are no longer sterile, are used to administer subsequent injections. For example, the World Health Organization (WHO) estimates that blood-borne diseases, such as Hepatitis and human immunodeficiency virus (HIV), are being transmitted due to reuse of such syringes, resulting the death of more than one million people each year.

As such, in response to this issue of reuse, single use injection needles have been made to prevent the possible reuse of such devices and reduce the spread of fatal blood-borne diseases. Although single use injection needles have reduced the potential spread of blood-borne diseases, such needles have drawbacks. For example, some single use injection needles are limited to delivering vaccines by the intramuscular or subcutaneous routes. Recently, there is renewed interest in intradermal vaccine delivery. This renewed interest is driven by the fact that the dermis and epidermis of human skin are rich in certain immune receptors and antigen-presenting cells, suggesting that delivery of vaccines to these layers, rather than to muscle or subcutaneous tissue, should be more efficient and induce protective immune responses with smaller amounts of vaccine antigen. Some current single use injection needles are not configured for effective intradermal delivery, and thus are unable to provide the benefits associated there with.

Through research, it has been found that intradermal delivery provides the potential for dose-sparing, in which a fraction of the typical dose of a vaccine is shown to be effective via the intradermal delivery route. The current project for poliovirus eradication is a leading example. The WHO is scheduling to phase out oral administration of the poliovirus vaccine by 2016 due to the fact that the oral varieties of the vaccine can mutate into an untreatable wild-type virus, which presents difficulty in fully eradicating the poliovirus. The WHO plans to use an inactivated poliovirus vaccine which has proven to be more effective than the oral variety, due in part to the requirement that the inactivated poliovirus be administered via intradermal delivery, thus requiring a device which could provide intradermal injection of a small dose (e.g., 0.05 ml to 0.1 ml).

A major problem, however, with intradermal delivery is the difficulty in precisely delivering the drug into the dermal layer. Generally, the outer layer, the epidermis, has a thickness of about 0.05 to 2 mm and the dermis has a thickness between about 1.5 and 4 mm. Thus, to deliver an agent to the dermis, the needle must penetrate the skin to a depth of no more than 5 mm, preferably between about 2 and 4 mm. In some rare instances, penetration may be upwards of 15 mm. It is very difficult to control an injection to this shallow depth.

One technique for administering intradermal injections is known as the Mantoux procedure. During such a procedure, fine gauge needle is inserted at about a 45 degree or less angle in an attempt to deliver the agent into the dermis. However, the Mantoux procedure is relatively complicated and requires technical skill from the medical professional or individual administering the injection. Additionally, the Mantoux procedure can prove painful for the individual receiving the injection, especially when a person without experience is administering the injection. Thus, the Mantoux procedure is not a preferred method, particularly in instances in which administration is occurring outside of a health facility and by a non-professional.

Some devices have been proposed for providing intradermal injections, which include shortened needles compared to conventional needle sizes. For example, micro patches, which may include skin-patches covered in micro needles coated with, or composed of, vaccine, have been proposed. However, such devices require special formulation and testing and trials of the vaccine, which may take many years to pass, as well as a large amount of funding. Additionally, other micro needle injection devices have been developed that include a prefilled dose of a vaccine, or other medicament. However, because the device is prefilled, and the vaccine typically must be maintained within a certain temperature range, the implementation of such prefilled injection devices can be costly, as such devices must be shipped and stored in accordance with the cold chain requirements. As such, such devices are not conducive to instances in which injections must be given to large numbers of individuals over a short period of time in areas outside of a health facility and without suitable storage facilities, as may be the case in developing countries.

### SUMMARY

The present invention provides a single use injection device that overcomes the drawbacks of current intradermal injection devices and methods. In particular, the single use injection device of the present invention is capable of intradermal delivery of an agent (e.g., vaccine, drug, medicament, etc.) in a controlled manner and without requiring specialized skill in administering delivery of such agent. The injection device is configured to be filled on-site and in the field with a microdose of an agent, while remaining sterile and preventing the potential for contamination during the filling process. Thus, because the injection device itself is not prefilled, the injection device of the present invention does not require the maintenance of a certain temperature (e.g., 2 to 8 degrees Celsius) during shipment or storage, thus cutting down on the overall costs. Rather than maintaining the injection device at a constant temperature, as is the case with current devices, only the source containing the vaccine or drug (e.g., single supply provided in filling syringe) need by maintained at a constant temperature.

Additionally, because the injection device is configured to store and deliver a microdose of agent, the injection device allows for dose-sparing. Dose-sparing may provide for a successful immunization program, particularly in resource-poor settings, by potentially reducing the per-injection cost (including transport and storage) of vaccines because more doses might be obtained from the existing vaccine presentation. Dose-sparing might also extend the availability of vaccines in cases where supply is limited by manufacturing capacity.

The injection device is configured to allow delivery of the agent to the patient in a relatively simple manner, without requiring specialized training for injecting a needle portion intradermally. In particular, the injection device is designed such that a person administering the agent (e.g., administrator) need only press the injection device against the administration site (e.g., shoulder, arm, chest, etc.), in which the device is configured such that needle penetration is limited to the correct length and orientation within the administration site. Upon needle penetration, the administrator then may fully compress a reservoir containing the micro dose of agent, thereby delivering the correct predefined dosage to the patient. Accordingly, the injection device of the present invention does not require a trained, skilled healthcare profession for administration of vaccines or drugs. As such, the injection device may be particularly useful in situations in which vaccines or drugs are being administered in non-healthcare related facilities (e.g., outside of clinics or hospitals) and given to large numbers of individuals over a short period of time by a non-professional. The injection device is further configured to be rendered incapable of reuse following its delivery of the agent to a patient, thereby preventing reuse of the device and reducing the risk of the spreading blood-borne diseases through reuse.

In one aspect, the present invention provides a single use injection device including a needle for intradermal injection of a fluid agent into a patient and a base member for providing the fluid agent into the needle. The base member includes a proximal end having an inlet port configured to receive the fluid agent from a source and a distal end having an outlet port coupled to the needle and configured to provide the fluid agent thereto. The base member further includes a channel providing a fluid pathway from the inlet port to the outlet port and a one-way valve positioned within the fluid pathway of the channel. The one-way valve is configured to limit fluid flow to an antegrade direction from the inlet port towards the outlet port.

The injection device further includes a top member coupled to the base member. The top member includes at least a compressible reservoir member in fluid communication with the fluid pathway of the channel. The reservoir member has an interior volume configured to receive and store fluid agent passing through the one-way valve and further configured to expel the fluid agent into the fluid pathway and through the outlet port into the needle in response to a compression force applied thereto. Accordingly, upon receiving a fluid agent from a source via the inlet port, the one-way valve is configured to only permit unidirectional flow of the fluid agent from the inlet port through the valve and towards the outlet port via the fluid pathway of the channel. Thus, when filling the injection device with a fluid agent stored in a filler syringe, for example, a person need only couple the syringe to the inlet port and then fill the reservoir with the fluid agent by applying pressure to a plunger of the filler syringe. Due to the one-way valve, the fluid agent is only permitted to flow within the reservoir and prevented from flowing in a retrograde fashion out of the reservoir. Furthermore, the interior volume of the reservoir may be within a range considered to be a micro dose. Accordingly, rather than requiring a person to closely monitor the exact amount of fluid agent provided to the injection device, they need only provide the fluid agent to the injection device until the interior volume of the reservoir is completely filled (the interior volume is limited to the dosage amount for any given fluid agent).

In some embodiments, a seal member may cover the inlet port of the base member so as to prevent any contaminants from entering the inlet port and potentially contaminating the injection device prior to filing the injection device with the fluid agent. For example, a single use seal member composed of a relatively thin sheet of material (e.g., metal foil, plastic, etc.) may be hermetically sealed to the opening of the inlet port, thereby preventing contaminants (e.g., gases, fluids, dirt, debris, etc.) from entering the injection device. The seal member is configured to rupture upon coupling of the filler syringe to the inlet port, thereby allowing a fluid to enter into the injection device via the inlet port. Accordingly, the seal member provides a measure of security to ensure that the injection device remains sterile until it is to be used. Accordingly, the seal member may be applied to the injection device prior, during, or post sterilization process, at which point the empty injection device may be shipped and stored at a desired location and will remain sterile, due, in part, to the seal member, thereby improving the process of storing such devices and the speed of assembly and use of such devices. Additionally, us of the seal member prevents the need additional packaging for the injection device, such as blister or ribbon packaging, thus providing a further economic advantage.

The injection device may be configured to prevent unintentional needle sticks, and thus reduce the potential for spreading blood-borne diseases. For example, in some embodiments, the base member further includes a needle protector member extending from distal end adjacent to the outlet port. The needle protector member is configured to move between a closed position, in which a penetrating tip of the needle is shielded, and an open position, in which the penetrating tip of the needle is exposed. Accordingly, the needle protector member may be in a closed position while the injection device is being shipped, stored, and handled (e.g., during filling of the injection device). An administrator need only move the needle protector member to an open position to expose the needle for delivering the fluid agent to a target site on a patient. Upon delivering the fluid agent, the administrator may then move the needle protector member to a closed position and discard the injection device, so as to prevent unintentional needle sticks.

As previously described, injection devices consisting with the present disclosure are not prefilled. Accordingly, rather than maintaining the individual injection device at a constant temperature, as is the case with some current devices, only the source (e.g. filler syringe or vial) containing the fluid agent need be maintained at a constant temperature. Additionally, because the reservoir member of the injection device is configured to store and expel a micro dose of the fluid agent, the injection device of the present invention allows for dose-sparing. Accordingly, a plurality of empty injection devices may be shipped and stored, at a reduced cost, and then filled directly on-site and on an as-needed basis, such that only a single filler syringe is required for hundreds of doses to be delivered at any given point. Additionally, because the injection device is not prefilled, it may be sterilized at any point prior to being filled with the fluid agent, which further improves the bulk shipping and storage of such devices.

The base member and top member may be formed of medical grade materials. In some embodiments, the base member and top member may be formed from a thermoplastic polymer, for example. An advantage of the construction of the injection device is that the base and top members may be produced separately from one another, wherein the base member may have a consistent production size and shape, while production of the top member may vary depending on the dosage amount. For example, certain vaccines require specific dosage amounts. Accordingly, a first production of top members can be produced so as to have a reservoir having an interior volume corresponding to a dosage amount recommended for a first vaccine (e.g., poliovirus vaccine) and a second production of top members can be produced so as to have a reservoir having an interior volume corresponding to dosage amount recommended for a second vaccine (e.g., Hepatitis). Accordingly, different dosage amounts can be easily produced (producing different top members) while still using a universal production of base members. The top member is then sealed to a base member to provide an assembled injection device.

The injection device is further configured to be rendered incapable of reuse following its delivery of the agent to a patient, thereby preventing reuse of the device and reducing the risk of the spreading blood-borne diseases through reuse. For example, in some embodiments, the reservoir member is configured to substantially collapse and reduce the interior volume upon substantial compression applied thereto. In particular, the top member may include an inelastic material such that the reservoir member is prevented from being reformed and the interior volume prevented from expanding subsequent to substantial compression. In some embodiments, the top member may further include a valve cover configured to substantially enclose the one-way valve within. Upon substantial compression applied to the valve cover, the valve cover is configured to substantially collapse upon the one-way valve and render the one-way valve inoperable, thereby blocking fluid flow from the inlet port to the reservoir member and disabling the injection potential.

### BRIEF DESCRIPTION OF THE DRAWINGS

FIG. 1 is a perspective exploded view of a single use injection device consistent with the present disclosure.

FIG. 2 is a top elevation view of the single use injection device of FIG. 1 illustrating the base and top members in an assembled state.

FIG. 3 is side view of the single use injection device of FIG. 1 illustrating the base and top members in an assembled state.

FIGS. 4 and 5 illustrate coupling of the single use injection device of FIG. 1 to a source for providing a fluid agent to the single use injection device.

FIGS. 6A and 6B illustrate intradermal delivery of a fluid agent with the single use injection device of FIG. 1.

### DETAILED DESCRIPTION

The present invention provides a single use injection device that is capable of intradermal delivery an agent (e.g., vaccine, drug, medicament, etc.) in a controlled manner and without requiring specialized skill in administering delivery of such agent. The injection device is further configured to be rendered incapable of reuse following its delivery of the agent to a patient, thereby preventing reuse of the device and reducing the risk of the spreading blood-borne diseases through reuse.

By way of overview, the present invention provides a single use injection device including a needle for intradermal injection of a fluid agent into a patient and a base member for providing the fluid agent into the needle. The base member includes a proximal end having an inlet port configured to receive the fluid agent from a source and a distal end having an outlet port coupled to the needle and configured to provide the fluid agent thereto. The base member further includes a channel providing a fluid pathway from the inlet port to the outlet port and a one-way valve positioned within the fluid pathway of the channel. The one-way valve is configured to limit fluid flow to an antegrade direction from the inlet port towards the outlet port.

The injection device further includes a top member coupled to the base member. The top member includes at least a compressible reservoir member in fluid communication with the fluid pathway of the channel. The reservoir member has an interior volume configured to receive and store fluid agent passing through the one-way valve and further configured to expel the fluid agent into the fluid pathway and through the outlet port into the needle in response to a compression force applied thereto.

The injection device is configured to allow delivery of the agent to the patient in a relatively simple manner, without requiring specialized training for injecting a needle portion intradermally. In particular, the injection device is designed such that it may be filled on-site and in the field with a microdose of an agent, while remaining sterile and preventing the potential for contamination during the filling process. For example, when filling the injection device with a fluid agent, a person need only couple a filler syringe or vial containing the fluid agent to the inlet port and then fill the reservoir with the fluid agent by applying pressure to a plunger of the filler syringe or some other filling method. Due to the one-way valve, the fluid agent is only permitted to flow within the reservoir and prevented from flowing in a retrograde fashion out of the reservoir. Furthermore, the interior volume of the reservoir may be within a range considered to be a micro dose. Thus, the injection device does not require exact measurements when filling the reservoir. Instead, a person need only completely fill the reservoir, which includes the correct dosage, and further prevents overfilling, as the interior volume is limited to the dosage amount for any given fluid agent.

Because the injection device itself is not prefilled, the injection device of the present invention does not require the maintenance of a certain temperature (e.g., 2 to 8 degrees Celsius) during shipment or storage, thus cutting down on the overall costs. Rather than maintaining the injection device at a constant temperature, as is the case with current devices, only the source containing the vaccine or drug (e.g., single supply provided in filling syringe or vial) need be maintained at a constant temperature. Additionally, because the injection device is configured to store and deliver a microdose of agent, the injection device allows for dose-sparing. Dose-sparing may provide for a successful immunization program, particularly in resource-poor settings, by potentially reducing the per-injection cost (including transport and storage) of vaccines because more doses might be obtained from the existing vaccine presentation. Dose-sparing might also extend the availability of vaccines in cases where supply is limited by manufacturing capacity. Accordingly, a plurality of empty injection devices may be shipped and stored, at a reduced cost, and then filled directly on-site and on an as-needed basis, such that only a single filler syringe is required for hundreds of doses to be delivered at any given point.

Once filled, the injection device is designed such that a person administering the agent (e.g., administrator) need only press the injection device against the administration site (e.g., shoulder, arm, chest, etc.), in which the device is configured such that needle penetration is limited to the correct length and orientation within the administration site. For example, in some embodiments, the needle is positioned substantially perpendicular relative to a plane along which the distal end of the base member lies, such that the needle is configured to be inserted into a patient's skin at a substantially perpendicular angle and the distal end is configured to contact the patient's skin indicating adequate depth of penetrating for intradermal injection of the fluid agent.

Upon needle penetration, the administrator then may fully compress a reservoir containing the micro dose of agent, thereby delivering the correct predefined dosage to the patient. The injection device is further configured to be rendered incapable of reuse following its delivery of the agent to a patient, thereby preventing reuse of the device and reducing the risk of the spreading blood-borne diseases through reuse. For example, in some embodiments, the reservoir member is configured to substantially collapse and reduce the interior volume upon substantial compression applied thereto. In particular, the top member may include an inelastic material such that the reservoir member is prevented from being reformed and the interior volume prevented from expanding subsequent to substantial compression. In some embodiments, the top member may further include a valve cover configured to substantially enclose the one-way valve within. Upon substantial compression applied to the valve cover, the valve cover is configured to substantially collapse upon the one-way valve and render the one-way valve inoperable, thereby blocking fluid flow from the inlet port to the reservoir member or rendering the device inoperable.

Furthermore, the injection device may be configured to prevent unintentional needle sticks, and thus reduce the potential for spreading blood-borne diseases. For example, in some embodiments, the base member further includes a needle protector member extending from distal end adjacent to the outlet port. The needle protector member is configured to move between a closed position, in which a penetrating tip of the needle is shielded, and an open position, in which the penetrating tip of the needle is exposed.

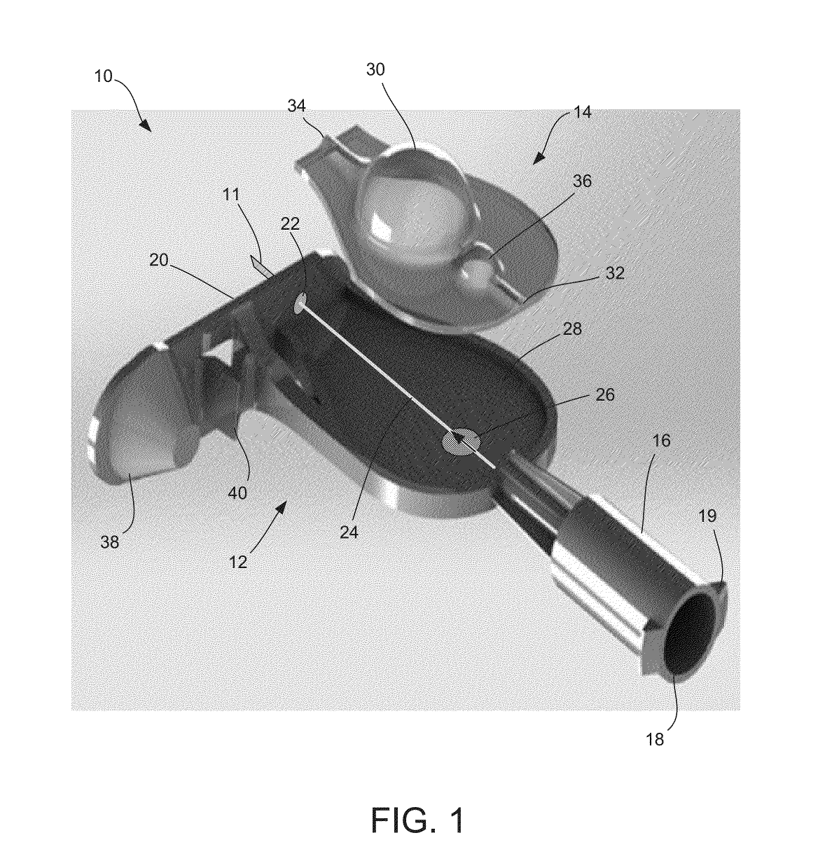

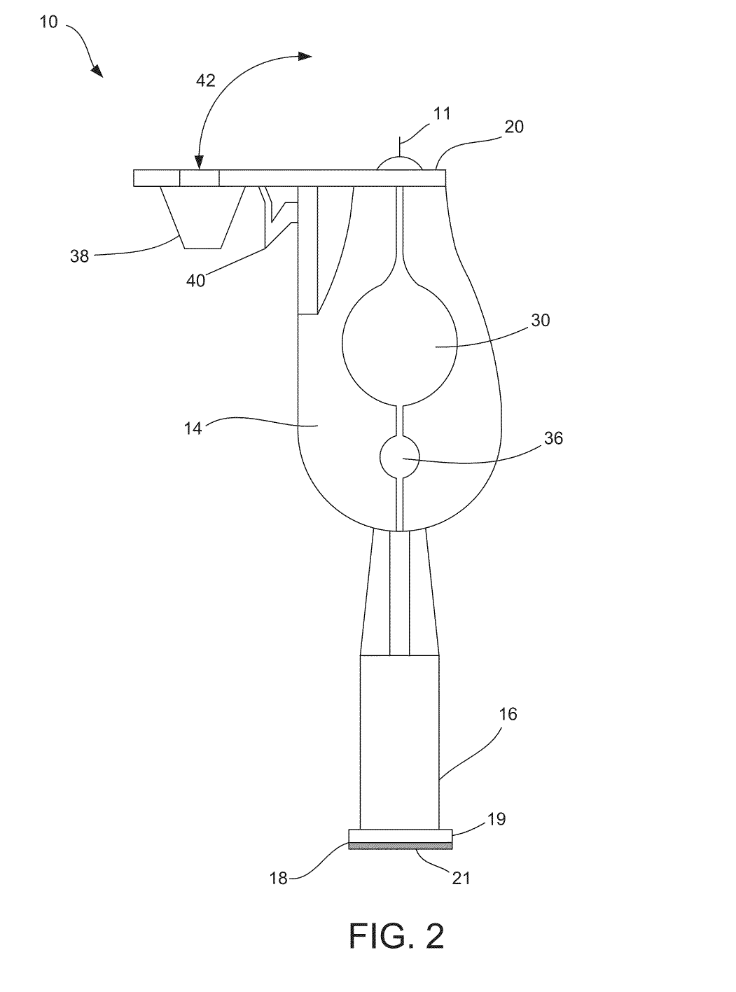

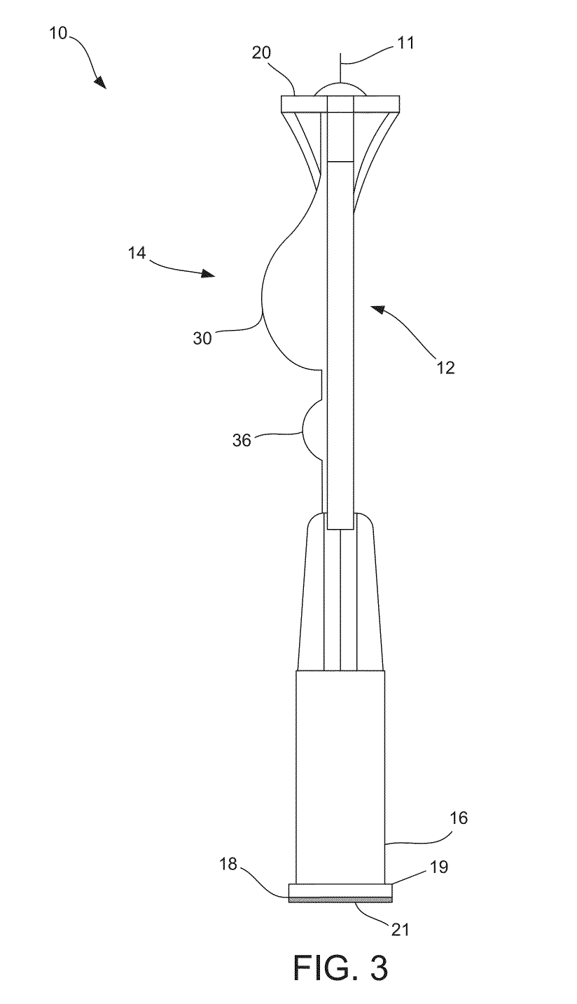

FIG. 1 is a perspective exploded view of a single use injection device 10 consistent with the present disclosure. FIGS. 2 and 3 are top and side elevation views of the single use injection device 10 of FIG. 1 in an assembled state. As shown, the single use injection device 10 includes a needle 11 having a tip configured for penetrating a target site and injecting a fluid agent into the target site. As will be described in greater detail herein, the needle may include a micro needle configured to penetrate a patient's skin down to a depth of the dermis and deliver a dosage of fluid agent thereto. The device 10 further includes a base member 12 and a top member 14 coupled thereto, wherein the combined base and top members 12, 14 are configured to provide the fluid agent into the needle for subsequent injection. As generally understood, the fluid agent may include any type of agent to be injected into a patient (e.g., mammal, either human or non-human) and capable of producing an effect. Accordingly, the agent may include, but is not limited to, a vaccine, a drug, a therapeutic agent, a medicament, or the like.

The base member 12 includes a proximal end 16 having an inlet port 18 configured to receive fluid agent from a source and a distal end 20 having an outlet port 22 coupled to the needle 11 and configured to provide the fluid agent thereto. As described in greater detail herein, the source of the fluid agent may include a filling syringe, for example, configured to be releasably coupled to the inlet port 18 of the base member 16. As shown, the inlet port 18 may include a Luer-type connection 19, such as a Luer-Lok fitting, configured to releasably engage a corresponding Luer-type connection on a hub of the syringe, thereby providing a fluid connection between the syringe and the inlet port 18 of the base member 12. It should be noted that the inlet port 18 need not be limited to an ISO standard (e.g. ISO 594) luer fitting. In other embodiments, the inlet port 18 may include non-standard connection fittings to be coupled with non-standard connection fitting of a source or adapter, for example.

As shown, a seal member 21 may cover the inlet port 18 so as to prevent any contaminants from entering the inlet port 18 and potentially contaminating the injection device 10 prior to filing the injection device 10 with the fluid agent. For example, a single use seal member 21 may be composed of a relatively thin sheet of material (e.g., metal foil, plastic, etc.) may be hermetically sealed to the opening of the inlet port 18, thereby preventing contaminants (e.g., gases, fluids, dirt, debris, etc.) from entering the injection device 10. The seal member 21 may be coupled to the inlet port 18 by any known sealing techniques (e.g., heat, vibration, or adhesive process). The seal member 21 is configured to be durable in the sense that it provides a sufficient seal with the inlet port 18 and prevent contaminants from entering into the device 10 via the inlet port 18 while also being configured to be pliable and rupture upon coupling of the inlet port 18 to a source (e.g., hub of filler syringe), thereby allowing a fluid to enter into the injection device 10 via the inlet port 18. Accordingly, the seal member 21 provides a measure of security to ensure that the injection device 10 remains sterile until it is to be used.

In some embodiments, the seal member 21 may be removed manually by hand, simply by gripping and pulling a tab (not shown) on a periphery of the seal member 21. In other embodiments, the seal member 21 could be incorporated into the base plate (not shown) used for transport and presentation of the injection device 10. Accordingly, upon removal of the injection device 10 from the base plate, the seal member 21 may break the seal with the inlet port 18, thereby exposing the inlet port 18 and allowing filling of the injection device 10.

The base member 12 may further include a channel 24 formed within a portion thereof and providing a fluid pathway from the inlet port 18 to the outlet port 22. Accordingly, upon receipt of fluid agent from a source, via the inlet port 18, the fluid agent may flow within the pathway provided by the channel 24. The base member 12 further includes a one-way valve 26 positioned within the fluid pathway of the channel 24. The one-way valve 26 is configured to permit antegrade flow of fluid from the inlet port 18 to the outlet port 22, while preventing retrograde flow (e.g., backflow) of fluid from the outlet port 22 through the valve 26 and through the inlet port 18. For example, the one-way valve 26 may include an open inlet end and an adjustable outlet end configured to move between a normally closed position and an open position. The one-way valve 26 is positioned such that the open inlet end is configured to receive fluid from the inlet port 18, and, upon sufficient application of fluid pressure in a direction away from the inlet port 18 and towards the outlet port 22 (e.g., depressing plunger of filling syringe to fill device 10 with fluid agent) the outlet end of the valve 26 moves from the normally closed position to an open position to allow fluid to flow therethrough in a direction towards the outlet port 22, as indicated by the directional arrow. However, when in a closed position, the outlet provides a substantially leak-proof and/or airtight seal so as to prevent any fluid from entering the valve 26 from the outlet end. Furthermore, the valve 26 is configured such that any application of fluid pressure in a direction away from the outlet port 22 and towards the outlet end of the valve 26, the outlet end remains closed, thereby preventing any fluid from flowing through the valve 26 in a retrograde direction from the outlet port 22 towards the inlet port 18. As generally understood, the one-way valve 26 may include any type of valve configured to permit fluid to flow only in a single direction. The one-way valve 26 may include any type of valve having medical grade material and configured to be used with the flow of fluids. For example, the one-way valve 26 may include a Reed valve or a Heimlich valve.

The top member 14 may be formed separately from the base member 12, which provides advantages, as previously described herein. Accordingly, the top member 14 may be coupled to a portion of the base member 12 along a mounting section 28. For example, the mounting section 28 generally includes a large portion of the base member 12 and includes at least a portion of the channel 24 and the one-way valve 26, such that, upon coupling the top member 14 to the mounting section 28 of the base member 12, the top member substantially encloses the channel 24 and the one-way valve 26.

The top member 14 includes a compressible reservoir member 30 and a compressible valve cover 26, such that, upon coupling the top member 14 to the base member 12, the reservoir member 30 is in fluid communication with the fluid pathway of the channel 24 and the valve cover 36 substantially encloses the one-way valve 26. The top member 14 may further include an inlet 32 and an outlet 34 and defining a fluid pathway extending there between and in fluid communication with the reservoir member 30 and valve cover 36. The one-way valve 26 and valve cover 36, if required, may be positioned within or in close proximity to the reservoir member 30. Accordingly, once coupled to the base member 12, the inlet 34 and outlet 34 and the pathway extending there between may substantially correspond to the fluid pathway of the channel 24, thereby cooperating with one another to form a combined single channel pathway from the inlet port 18 to the outlet port 22.

The top member 14 may be coupled to the base member 12 by any known means so as to create a hermetic seal. For example, the base and top members 12, 14 may be sealed with one another via any known adhesives, cements, ultrasonic welding, or thermoplastic bonding techniques. The base and top members 12, 14 are composed of a medical grade material. In some embodiments, the base member 12, the top member 14, or both, may be composed of a thermoplastic polymer, including, but not limited to, polypropylene, polyethylene, polybenzimidazole, acrylonitrile butadiene styrene (ABS) polystyrene, polyvinyl chloride, PVC, or the like.

The reservoir member 30 includes an interior volume configured to receive and store a fluid agent passing through the one-way valve 26. Upon applying a compression force to the reservoir member 30, the fluid agent is expelled into the fluid pathway of the channel 24 and through the outlet port 22 into the needle 11. Accordingly, the method of delivering the fluid agent into a patient is a relatively simple and straightforward process which simply requires an administrator to apply sufficient pressure to the filled reservoir member 30 so as to deform the reservoir, resulting in expulsion of the stored fluid agent from the interior volume. Due to the one-way valve 26, the fluid agent is force to flow in a direction towards the outlet port 22 and out of the needle 11.

The base member 12 further includes a needle protector member 38 extending from the distal end 20 and adjacent to the outlet port 22. The needle protector member 38 may be coupled to the distal end 20 by way of any known means. In the illustrated embodiment, the needle protector member 38 is coupled to the distal end 20 by way of a living hinge 40, for example. Accordingly, the needle protector member 38 is configured to move between a closed position and an open position, as indicated by arrow 42. When in a closed position, the needle protector member 38 is configured to substantially enclose the penetrating tip of the needle 11, thereby shielding one from inadvertent needle sticks. When in an open position, as shown, the penetrating tip of the needle 11 is exposed and ready for intradermal injection on a target site of a patient. Accordingly, the needle protector member 38 may be in a closed position while the injection device 10 is being shipped, stored, and handled (e.g., during filling of the injection device 10). An administrator need only move the needle protector member 38 to an open position to expose the needle 11 for delivering the fluid agent to a target site on a patient. Upon delivering the fluid agent, the administrator may then move the needle protector member 38 to a closed position and discard the injection device 10, so as to prevent unintentional needle sticks. It should be noted that, depending on the length of needle 11, the needle protector member 38 shape or size may differ so as to adequately enclose the needle 11 within.

The injection device is configured to allow delivery of the agent to the patient in a relatively simple manner, without requiring specialized training for injecting a needle portion intradermally. In particular, the injection device is designed such that it may be filled on-site and in the field with a microdose of an agent, while remaining sterile and preventing the potential for contamination during the filling process.

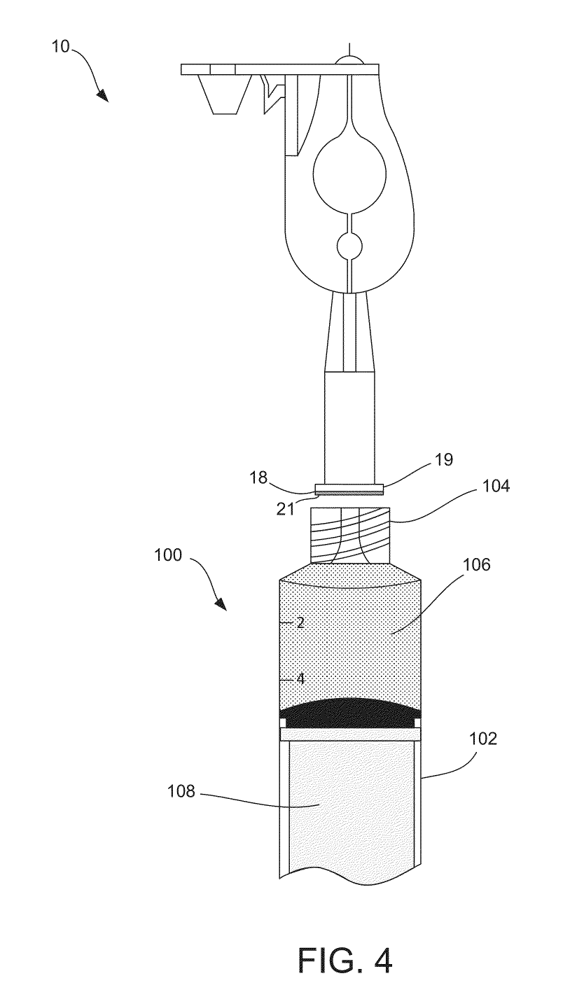

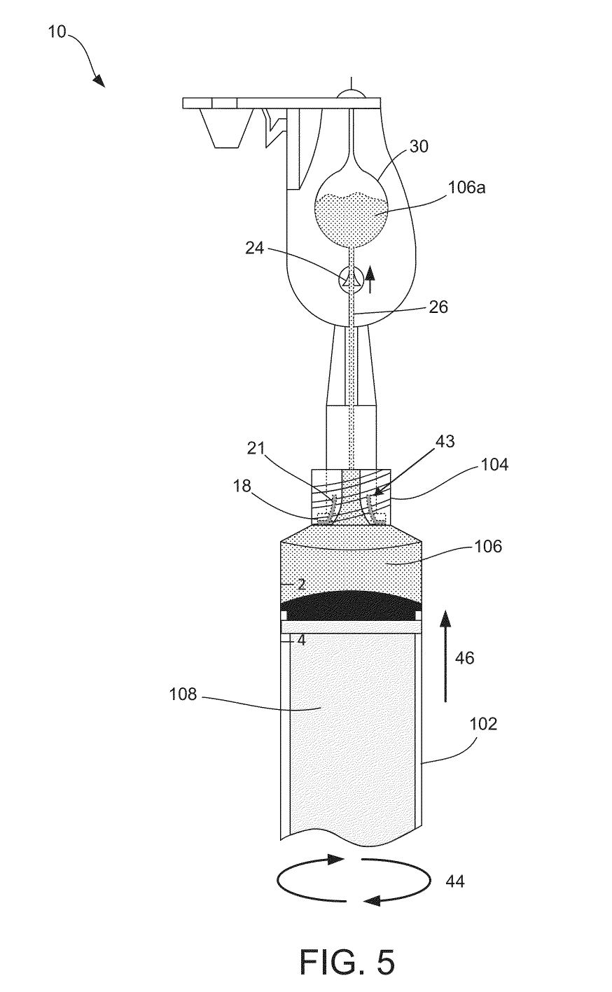

For example, FIGS. 4 and 5 illustrate coupling of the single use injection device 10 to a source for dispensing a fluid agent into the injection device 10. In the illustrated embodiment, the source may include a filler syringe 100, for example. The filler syringe 100 may be embodied as a conventional syringe. Accordingly, the filler syringe 100 includes a barrel 102 having a distal hub 104 configured to be releasably coupled to the inlet port 18 of the base member 12 of the injection device 10. For example, the inlet port 18 may include a Luer-type connection 19, such as a Luer-Lok fitting, configured to releasably engage a corresponding Luer-type connection on the hub 104 of the syringe 100, thereby providing a fluid connection between the interior volume of the barrel 102 of the syringe 100 and the inlet port 18 and subsequent fluid pathway formed by the channel 24 of the base member 12.

In order to filling the injection device 10, specifically the reservoir member 30, with a fluid agent 106 contained with the syringe 100, a person need only couple the hub 104 with the inlet port 18. As shown in FIG. 4, the seal member 21 is intact and covering the inlet port 18 so as to prevent any contaminants from entering the inlet port 18 and potentially contaminating the injection device 10 prior to filing the injection device 10 with the fluid agent. Upon inserting the hub 104 into engagement with the inlet port 18, the hub 104 is configured to pierce the seal member 21, upon which the seal member 21 ruptures and tears, as indicated by arrow 43, thereby breaking the hermetic seal and allowing fluid to be providing from the syringe 100 into the device 10 through the inlet port 18. For example, upon rotating either the syringe 100 or device 10, as indicated by arrow 44, the hub 104 and inlet port 18 may contact and come into threaded engagement. A person may then fill the reservoir 40 with the fluid agent 106 by applying pressure to a plunger 108 of the filler syringe 100, as indicated by arrow 46. Due to the one-way valve 26, the fluid agent 106 is only permitted to flow in a direction towards the reservoir 30 and prevented from flowing in a retrograde fashion out of the reservoir 30. Furthermore, the interior volume of the reservoir 30 may be within a range considered to be a micro dose, such as 0.05 ml to 1.0 ml. Accordingly, the injection device 10 does not require exact measurements when filling the reservoir 30. Instead, a person need only completely fill the reservoir, which includes the correct dosage, and, once completely filled, the correct dosage has been reached and the buildup of pressure will prevent the plunger 108 from advancing further. Accordingly, the device 10 allows consistent filling and dosing of the fluid agent 106 from device to device (e.g., filling up tens of hundreds of devices 10 at any one time). Accordingly, when in the field or directly on-site, a person may use a single filling syringe 100 to fill a plurality of empty injection devices 10 in a consistent manner. The filling syringe 100 essentially acts as a means of storing and dispensing aliquots of the fluid agent.

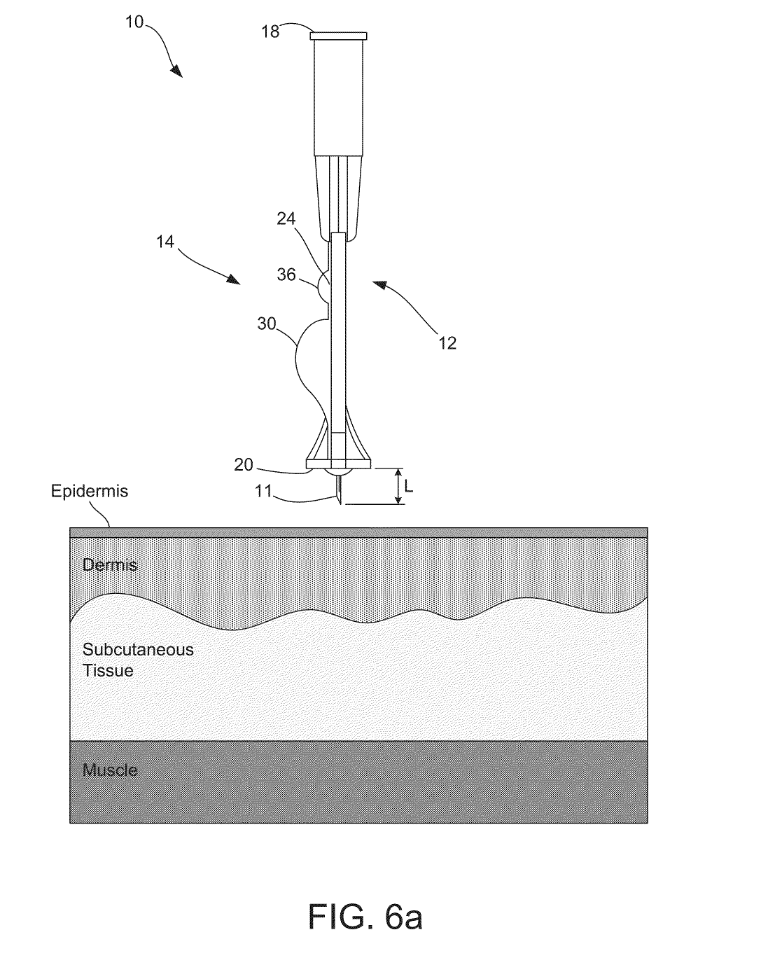

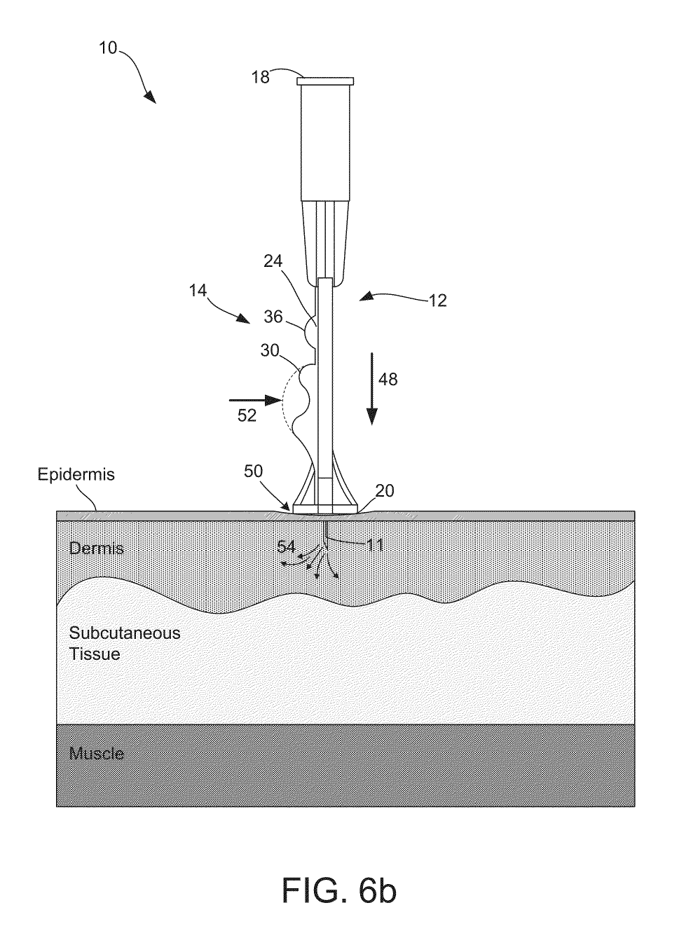

Once filled, the injection device 10 is designed such that a person administering the agent (e.g., administrator) need only press the injection device 10 against the administration site (e.g., shoulder, arm, chest, etc.). FIGS. 6A and 6B illustrate intradermal delivery of a fluid agent with the single use injection device of FIG. 1. As shown, the injection device 10 is configured to allow delivery of the agent to the patient in a relatively simple manner, without requiring specialized training for injecting a needle portion intradermally. In particular, the injection device is designed such that a person administering the agent (e.g., administrator) need only press the injection device against the administration site (e.g., shoulder, arm, chest, etc.), in which the device is configured such that needle penetration is limited to the correct length and orientation within the administration site. As shown, the injection device 10 may be removed from the filler syringe 100 and used to administer the fluid agent as a standalone device. However, it should be noted that the injection device 10 may remain coupled to the filler syringe 100 during administration of the fluid agent, such that an administrator may use the filler syringe 100 as a handle or means of stabilizing the injection device 10 during delivery of the fluid agent to a patient.

As shown in FIG. 6A, the needle 11 is positioned substantially perpendicular relative to a plane along which the distal end 20 of the base member 12 lies, such that the needle 11 is configured to be inserted into a patient's skin at a substantially perpendicular angle. This is a much more straightforward process for intradermal delivery of an agent, particularly when compared to the Mantoux procedure. Furthermore, the distal end is configured to contact the patient's skin during penetration of the needle 11, thereby indicating adequate depth of penetrating for intradermal injection of the fluid agent. For example, the needle 11 may be a micro-needle having a length L (measured from the distal end 20) in the range of 0.5 mm to 4 mm. It should be noted that in some embodiments, the needle 11 may have a length L upwards of 15 mm to 20 mm.

Accordingly, as shown in FIG. 6B, upon an administrator applying pressure in a direction towards the target site, as indicated by arrow 48, the needle 11 is configured to penetrate the epidermis and dermis layers of skin. Upon sufficient contact between the distal end of the base member 12 and the outer layer of skin, as indicated by arrow 50, the needle 11 has achieved adequate penetration into the dermis for intradermal injection of the fluid agent. Upon the needle 11 reaching the adequate depth into the dermis, the administrator may then compress the reservoir member 30 containing the dosage of fluid agent so as to deliver the fluid agent into the dermis. For example, the reservoir member 30 is configured to substantially collapse and reduce the interior volume upon substantial compression applied thereto, as indicated by arrow 52. An administrator need only fully compress the reservoir member 30 so as to expel to required dosage. Upon compression of the reservoir member 30, the fluid agent is expelled into the fluid pathway of the channel 24 and out of the outlet port 22 and out of the needle 11, resulting in delivery of the fluid agent into the dermis, as indicated by arrow 54.

In some embodiments, the reservoir member 30 is shaped or sized such that, upon compression applied thereto, the reservoir member 30 is prevented from being reformed and the interior volume is prevented from expanding subsequent to substantial compression. Additionally, or alternatively, the valve cover 36 may be shaped or sized such that, upon compression applied thereto, the valve cover 36 is configured to substantially collapse upon the one-way valve 26 and render the one-way valve 26 inoperable, thereby blocking fluid flow into or out of the one-way valve 26. Accordingly, the injection device 10 configured to be rendered incapable of reuse following its delivery of the agent to a patient, thereby preventing reuse of the device and reducing the risk of the spreading blood-borne diseases through reuse.

Accordingly, the injection device 10 of the present invention does not require a trained, skilled healthcare profession for administration of vaccines or drugs. As such, the injection device may be particularly useful in situations in which vaccines or drugs are being administered in non-healthcare related facilities (e.g., outside of clinics or hospitals) and given to large numbers of individuals over a short period of time by a non-professional.

While several embodiments of the present disclosure have been described and illustrated herein, those of ordinary skill in the art will readily envision a variety of other means and/or structures for performing the functions and/or obtaining the results and/or one or more of the advantages described herein, and each of such variations and/or modifications is deemed to be within the scope of the present disclosure. More generally, those skilled in the art will readily appreciate that all parameters, dimensions, materials, and configurations described herein are meant to be exemplary and that the actual parameters, dimensions, materials, and/or configurations will depend upon the specific application or applications for which the teachings of the present disclosure is/are used.

Those skilled in the art will recognize, or be able to ascertain using no more than routine experimentation, many equivalents to the specific embodiments of the disclosure described herein. It is, therefore, to be understood that the foregoing embodiments are presented by way of example only and that, within the scope of the appended claims and equivalents thereto, the disclosure may be practiced otherwise than as specifically described and claimed. The present disclosure is directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the scope of the present disclosure.

All definitions, as defined and used herein, should be understood to control over dictionary definitions, definitions in documents incorporated by reference, and/or ordinary meanings of the defined terms.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Other elements may optionally be present other than the elements specifically identified by the “and/or” clause, whether related or unrelated to those elements specifically identified, unless clearly indicated to the contrary.

Reference throughout this specification to “one embodiment” or “an embodiment” means that a particular feature, structure, or characteristic described in connection with the embodiment is included in at least one embodiment. Thus, appearances of the phrases “in one embodiment” or “in an embodiment” in various places throughout this specification are not necessarily all referring to the same embodiment. Furthermore, the particular features, structures, or characteristics may be combined in any suitable manner in one or more embodiments.

The terms and expressions which have been employed herein are used as terms of description and not of limitation, and there is no intention, in the use of such terms and expressions, of excluding any equivalents of the features shown and described (or portions thereof), and it is recognized that various modifications are possible within the scope of the claims. Accordingly, the claims are intended to cover all such equivalents.

### INCORPORATION BY REFERENCE

References and citations to other documents, such as patents, patent applications, patent publications, journals, books, papers, web contents, have been made throughout this disclosure. All such documents are hereby incorporated herein by reference in their entirety for all purposes.

### EQUIVALENTS

Various modifications of the invention and many further embodiments thereof, in addition to those shown and described herein, will become apparent to those skilled in the art from the full contents of this document, including references to the scientific and patent literature cited herein. The subject matter herein contains important information, exemplification and guidance that can be adapted to the practice of this invention in its various embodiments and equivalents thereof.

What is claimed is:

1. A single use injection device comprising:
a needle for intradermal injection of a fluid agent into a patient;
a base member for providing said fluid agent into said needle, said base member comprising:
a proximal end having an inlet port configured to receive said fluid agent from a source and a distal end having an outlet port coupled to said needle and configured to provide said fluid agent thereto;
a channel providing a fluid pathway from said inlet port to said outlet port; and
a one-way valve positioned within said fluid pathway of said channel, said one-way valve configured to limit fluid flow to an antegrade direction from said inlet port towards the outlet port; and
a top member coupled to said base member and comprising a compressible reservoir member in fluid communication with said fluid pathway of said channel, said reservoir member having an interior volume configured to receive and store said fluid agent passing through said one-way valve and configured to expel said fluid agent into said fluid pathway and through said outlet port into said needle in response to a compression force applied thereto.

2. The single use injection device of claim 1, wherein said reservoir member is configured to substantially collapse and reduce said interior volume upon substantial compression applied thereto.

3. The single use injection device of claim 2, wherein said top member comprises an inelastic material such that said reservoir member is prevented from being reformed and said interior volume prevented from expanding subsequent to substantial compression.

4. The single use injection device of claim 1, wherein said base member further comprises a needle protector member extending from distal end adjacent to said outlet port and configured to move between a closed position, in which a penetrating tip of said needle is shielded, and an open position, in which said penetrating tip of said needle is exposed.

5. The single use injection device of claim 4, wherein said needle protector member is coupled to said distal end via a living hinge.

6. The single use injection device of claim 1, wherein said top member further comprises a valve cover configured to substantially enclose said one-way valve.

7. The single use injection device of claim 6, wherein, upon substantial compression applied to said valve cover, said valve cover is configured to substantially collapse upon said one-way valve and render said one-way valve inoperable, thereby blocking fluid flow from said inlet port to said reservoir member or rendering the injection device inoperable.

8. The single use injection device of claim 1, wherein said inlet port of said base member is configured to be releasably coupled to said source by a standard fluid fitting.

9. The single use injection device of claim 8, wherein said inlet port comprises a Luer-type connection configured to releasably engage a corresponding Luer-type connection of a syringe configured to provide said fluid agent.

10. The single use injection device of claim 1, wherein said needle is positioned substantially perpendicular relative to a plane along which said distal end of said base member lies, such that said needle is configured to be inserted into a patient's skin at a substantially perpendicular angle and said distal end is configured to contact said patient's skin indicating adequate depth of penetrating for intradermal injection of said fluid agent.

11. The single use injection device of claim 1, wherein each of said base member and said top member comprises a medical grade material.

12. The single use injection device of claim 1, wherein said top member comprises a thermoplastic polymer.

13. The single use injection device of claim 1, wherein said fluid agent is a vaccine.

14. An intradermal single use injection device comprising:
a needle for intradermal injection of a fluid agent into a patient;
a base member for delivering said fluid agent into said needle, said base member comprising:
a channel having an inlet and an outlet, said inlet configured to receive said fluid agent from a source and said outlet configured to provide said fluid agent to said needle;
a one-way valve positioned within said channel and configured to limit fluid flow in a direction from said inlet towards said outlet;
a needle protector member positioned adjacent to said outlet port and configured to move between a closed position, in which a penetrating tip of said needle is shielded, and an open position, in which said penetrating tip of said needle is exposed; and
a top member sealed to said base member, said top member comprising:
a compressible valve cover substantially enclosing said one-way valve; and
a reservoir member positioned adjacent to said one-way valve and in fluid communication with said channel, said reservoir member configured to store fluid agent passing through said one way valve and expel stored fluid agent in response to a compression force applied thereto so as to deliver said fluid agent to said needle for intradermal injection.

15. The intradermal single use injection device of claim 13, wherein, upon substantial compression of said reservoir member, said reservoir member is configured to collapse, thereby reducing an interior volume within and expelling a substantial amount of fluid agent stored within.

16. The intradermal single use injection device of claim 14, wherein said top member comprises an inelastic material such that said reservoir member is prevented from being reformed and said interior volume prevented from expanding subsequent to substantial compression.

17. The intradermal single use injection device of claim 13, wherein, upon substantial compression of said valve cover, said valve cover is configured to substantially collapse upon said one-way valve and render said one-way valve inoperable, thereby blocking fluid flow from said inlet to said reservoir member.

18. The intradermal single use injection device of claim 13, wherein said needle is a micro-needle having a length in the range of 0.5 mm to 10 mm.

19. The intradermal single use injection device of claim 13, wherein an interior volume of said reservoir member is in the range of 0.05 ml to 2.0 ml.

20. A single use injection system comprising:
a syringe for storing and dispensing aliquots of a fluid agent; and
a plurality of single use injection devices configured to receive aliquots of said fluid agent from said syringe, each of said single use injection devices comprises:
a needle for intradermal injection of an aliquot of fluid agent into a patient;
a base member for delivering said fluid agent into said needle, said base member comprising:
a channel having an inlet and an outlet, said inlet configured to releasably couple to said syringe and receive said aliquot of fluid agent and said outlet configured to provide said aliquot fluid agent to said needle; and
a one-way valve positioned within said channel and configured to limit fluid flow through said channel in a direction from said inlet towards said outlet;
a top member sealed to said base member, said top member comprising:
a reservoir member positioned adjacent to said one-way valve and in fluid communication with said channel, said reservoir member configured to store said aliquot of fluid agent passing through said one way valve and expel stored fluid agent in response to a compression force applied thereto so as to deliver said aliquot of fluid agent to said needle for intradermal injection.

21. The single use injection system of claim 20, wherein, upon substantial compression of said reservoir member, said reservoir member is configured to collapse, thereby reducing an interior volume within and expelling a substantial amount of said aliquot of fluid agent stored within.

22. The single use injection system of claim 21, wherein said top member comprises an inelastic material such that said reservoir member is prevented from being reformed and said interior volume prevented from expanding subsequent to substantial compression.

23. The single use injection system of claim 20, wherein said inlet of said base member comprises a Luer-type connection configured to releasably engage a corresponding Luer-type connection of a hub of said syringe.

24. The single use injection system of claim 20, wherein said base member further comprises a needle protector member adjacent to said outlet and configured to move between a closed position, in which a penetrating tip of said needle is shielded, and an open position, in which said penetrating tip of said needle is exposed.

25. The single use injection system of claim 20, wherein said fluid agent is a vaccine.

26. The single use injection system of claim 20, wherein said patient is a mammal.
